(19) United States
(12) Patent Application Publication
(10) Pub. No.: US 2024/0277916 A1
(43) Pub. Date: Aug. 22, 2024

(54) WEARABLE COMPRESSED FOAM GENERATED NEGATIVE PRESSURE THERAPY DEVICES, SYSTEMS, AND METHODS

(71) Applicant: KCI Manufacturing Unlimited Company, Westmeath (IE)

(72) Inventors: Andrew J. MCGREGOR, San Antonio, TX (US); Amy K. MCNULTY, San Antonio, TX (US); Tyler J. SANDBACK, San Antonio, TX (US)

(21) Appl. No.: 18/694,361

(22) PCT Filed: Aug. 23, 2022
(86) PCT No.: PCT/IB2022/057875

§ 371 Date: Mar. 21, 2024

Related U.S. Application Data
(60) Provisional application No. 63/250,591, filed on Sep. 30, 2021.

(51) Int. Cl. A61M 1/00

(52) U.S. Cl. CPC A61M 1/912; A61M 1/80; A61M 1/915

(57) ABSTRACT

A system for negative pressure therapy may include a dressing, a pump, and a pump actuation assembly. The dressing may be configured to be placed adjacent to a tissue site. The pump may be configured to be positioned in fluid communication with the dressing and to provide negative pressure to the dressing when the pump is activated. The pump may include a biasing element and a pump housing. The pump actuation assembly may include a sacrificial seal and an actuation device. The actuation device may be configured to activate the pump when the sacrificial seal is broken. Other dressings, systems, and methods are disclosed.

### CROSS-REFERENCE TO RELATED APPLICATIONS

This application is a U.S. National Stage Entry of PCT/IB2022/057875, filed Aug. 23, 2022, which claims the benefit, under 35 U.S.C. § 119(c), of U.S. Provisional Application No. 63/250,591, entitled “WEARABLE COMPRESSED FOAM GENERATED NEGATIVE PRESSURE THERAPY DEVICES, SYSTEMS, AND METHODS,” filed Sep. 30, 2021, each of which are incorporated herein by reference for all purposes.

### FIELD

This disclosure relates generally to medical treatment systems and, more particularly, but not by way of limitation, to dressings, devices, systems, and methods for treating a tissue site with reduced pressure.

### BACKGROUND

Depending on the medical circumstances, reduced pressure may be used for, among other things, reduced-pressure therapy to encourage granulation at a tissue site, draining fluids at a tissue site, closing a wound, reducing edema, promoting perfusion, and fluid management. Common dressings, systems, and methods may be susceptible to leaks and blockage that can cause a reduction in the efficiency of the therapy or a complete loss of therapy. Such a situation can occur, for example, if the amount of fluid in the dressing or system exceeds the fluid capacity of the dressing or system. Further, rigid components, vibrating electronic pumps and loose tubing common to some systems can contribute to patient discomfort and ultimately treatment noncompliance. Thus, improvements to dressings, devices, systems, and methods that enhance one or more non-limiting factors, such as fluid management, reliability, efficiency, patient comfort, and treatment compliance are desirable.

### SUMMARY

Shortcomings with certain aspects of tissue treatment dressings, devices, systems, and methods are addressed as shown and described in a variety of illustrative, non-limiting example embodiments herein.

In some example embodiments, a system for negative-pressure therapy is described. The system can include a dressing that can be configured to be placed adjacent to a tissue site, a pump that can be configured to be positioned in fluid communication with the dressing, and a pump actuation assembly. The pump can include a biasing element and a pump housing. The pump can be configured to provide negative pressure to the dressing when the pump is activated. The pump actuation assembly can include a sacrificial seal and an actuation device. The actuation device can be configured to activate the pump when the sacrificial seal is broken.

The biasing element can be configured to generate a variable volume within the pump housing when the pump is activated. In some examples, the biasing element can comprise foam and the pump housing can comprise a film that defines at least a portion of a sealed chamber that encases the foam. The pump housing can also comprise at least one check valve. The pump housing can be configured to permit re-activation of the pump to maintain a desired negative-pressure at the dressing. The pump and the actuation device can be non-electronic elements. The foam can have a first volume when it is in a compressed state prior to activation and a second volume after activation. The second volume can be greater than the first volume.

In some examples, the sacrificial seal can include a film layer with a zone of weakness, and the actuation device can include at least one pull tab. The at least one pull tab can be coupled to the film layer or formed integrally with the film layer. The at least one pull tab can be configured to stretch the film layer to form an opening in the film layer at the zone of weakness and to activate the pump when pulled. The sacrificial seal can be moveable from a sealed state to an activated state. The sacrificial seal can be closed in the sealed state. The sacrificial seal can be open in the activated state and the pump can be activated in the activated state. The opening can be formed through the sacrificial seal in the activated state when the film layer is stretched to failure at the zone of weakness.

The example system can further include a port defined through the pump actuation assembly. The port can be configured to enable fluid communication between the dressing and the pump. The sacrificial seal can be positioned in the port of the pump actuation assembly between a pump mounting portion and an external mounting portion. The pump mounting portion and the external mounting portion can each comprise a ring of closed cell foam.

In some examples, the pump can be coupled to the pump actuation assembly at the pump mounting portion of the pump actuation assembly. The pump actuation assembly can further include the external mounting portion opposite from the pump mounting portion. An exterior surface of the external mounting portion can carry a releasable adhesive. In some example embodiments, the pump can be a first pump and the pump actuation assembly can be a first pump actuation assembly. The system can further include at least a second pump and a second pump actuation assembly. One of the first pump or the second pump can be configured to be removed after the dressing is drawn down to a desired negative pressure.

Other example embodiments describe a pump module for use in a negative-pressure therapy system. The pump module can include a pump and a pump actuation assembly. Some examples of the pump can include a biasing element and a pump housing. Some examples of the pump actuation assembly can include a sacrificial seal and an actuation device that can be configured to activate the pump when the sacrificial seal is broken.

The biasing element can be configured to generate a variable volume within the pump housing when the pump is activated. Examples of the biasing element can comprise foam, and examples of the pump housing can comprise a film that defines at least a portion of a sealed chamber that encases the foam. The pump housing can also comprise at least one check valve. The pump housing can be configured to permit re-activation of the pump to maintain a desired negative-pressure at a dressing. The pump and the actuation device can be non-electronic elements. The foam can have a first volume when it is in a compressed state prior to activation and a second volume after activation. The second volume can be greater than the first volume.

In some examples, the sacrificial seal can include a film layer with a zone of weakness, and the actuation device can include at least one pull tab. The at least one pull tab can be coupled to the film layer or formed integrally with the film layer. The at least one pull tab can be configured to stretch the film layer to form an opening in the film layer at the zone of weakness and to activate the pump when pulled. The sacrificial seal can be moveable from a sealed state to an activated state. The sacrificial seal can be closed in the sealed state. The sacrificial seal can be open in the activated state and the pump can be activated in the activated state. The opening can be formed through the sacrificial seal in the activated state when the film layer is stretched to failure at the zone of weakness.

The example pump module can further include a port defined through the pump actuation assembly. The sacrificial seal can be positioned in the port of the pump actuation assembly between a pump mounting portion and an external mounting portion. The pump mounting portion and the external mounting portion can each comprise a ring of closed cell foam.

In some examples, the pump can be coupled to the pump actuation assembly at the pump mounting portion of the pump actuation assembly. The pump actuation assembly can further include the external mounting portion opposite from the pump mounting portion. An exterior surface of the external mounting portion can carry a releasable adhesive. In some embodiments, the pump can be a first pump and the pump actuation assembly can be a first pump actuation assembly. Some examples of the pump module can further include at least a second pump and a second pump actuation assembly. One of the first pump or the second pump can be configured to be removed after the dressing is drawn down to a desired negative pressure.

Illustrative example embodiments of a method for generating negative-pressure are also described herein. In some examples, a dressing can be provided that can be configured to be placed adjacent to a tissue site. Further, a pump module can be provided that can be configured to be placed in fluid communication with the dressing and to provide negative pressure to the dressing when activated. The pump module can comprise a pump and a pump actuation assembly. In some examples, the pump can be activated by breaking a sacrificial seal of the pump actuation assembly to permit the pump to draw fluids from the dressing. Breaking the sacrificial seal of the pump actuation assembly can include, for example, pulling at least one pull tab of an actuation device of the pump actuation assembly to break the sacrificial seal at a zone of weakness.

In some examples, a port of the pump actuation assembly can be coupled to the pump and to the dressing. The port can be configured to enable fluid communication between the dressing and the pump. In other embodiments, the port of the pump actuation assembly can be coupled to the pump and to a conduit that can be configured to be coupled in fluid communication between the port and the dressing. In some embodiments, a pump housing of the pump can be pinched after the pump has reached an expanded state in order to re-activate the pump.

In some examples, the pump can be coupled to the pump actuation assembly at a pump mounting portion of the pump actuation assembly. The pump actuation assembly can further comprise an external mounting portion that can be positioned opposite from the pump mounting portion. An external surface of the external mounting portion can carry a releasable adhesive. In some examples, the pump can be a first pump and the pump actuation assembly can be a first pump actuation assembly. At least a second pump and a second pump actuation assembly can also be provided in some examples. The first pump can optionally be removed from the pump module after the dressing is drawn to a desired negative pressure. The second pump can be activated before or after the first pump is removed from the pump module, or at a suitable time when the dressing is drawn to a desired negative pressure.

Other aspects, features, and advantages of the illustrative embodiments will become apparent with reference to the drawings and detailed description that follow.

### BRIEF DESCRIPTION OF THE DRAWINGS

FIG. 1 is a cut-away view of an illustrative embodiment of a system for treating a tissue site depicting an illustrative example embodiment of a pump module and a dressing deployed at a tissue site;

FIG. 2 is a cut-away view of the dressing of FIG. 1;

FIG. 3 is detail view taken at reference FIG. 3, depicted in FIG. 1, illustrating the dressing of FIG. 1 positioned proximate to tissue surrounding the tissue site;

FIG. 4A is an exploded view of the dressing of FIG. 1, depicted with an illustrative example embodiment of a release liner for protecting the dressing prior to application at a tissue site;

FIG. 4B is a plan view of an illustrative example embodiment of a base layer depicted in the dressing of FIG. 4A;

FIG. 5A is a cut-away view of an illustrative example pump and an illustrative example pump actuation assembly of the system of FIG. 1, shown in a compressed state prior to activation;

FIG. 5B is a cut-away view of the illustrative example pump and pump actuation assembly of FIG. 5A, shown in an expanded state after activation;

FIG. 6 is a cut-away view of the example pump actuation assembly of the pump module of FIG. 5;

FIG. 7A is a top view of the example pump actuation assembly of FIG. 6, illustrating an example embodiment of a sacrificial seal in a closed or sealed state;

FIG. 7B is a top view of the example pump actuation assembly of FIG. 6, illustrating an example embodiment of a sacrificial seal in an open or activated state;

FIG. 8 is a cut-away view of an illustrative example embodiment of a conduit interface connected to the pump;

FIG. 9 is a cut-away view of an illustrative example embodiment of the conduit interface connected to the dressing and the pump;

FIG. 10 is a perspective view of another example embodiment of the system including multiple pumps; and

FIG. 11 is a perspective view of the system of FIG. 10 illustrating additional details that may be associated with some example embodiments.

### DETAILED DESCRIPTION OF ILLUSTRATIVE EMBODIMENTS

In the following detailed description of non-limiting, illustrative embodiments, reference is made to the accompanying drawings that form a part hereof. Other embodiments may be utilized, and logical, structural, mechanical, electrical, and chemical changes may be made without departing from the scope of the appended claims. To avoid detail not necessary to enable those skilled in the art to practice the embodiments described herein, the description may omit certain information known to those skilled in the art. The following detailed description is non-limiting, and the scope of the illustrative embodiments are defined by the appended claims. As used herein, unless otherwise indicated, “or” does not require mutual exclusivity.

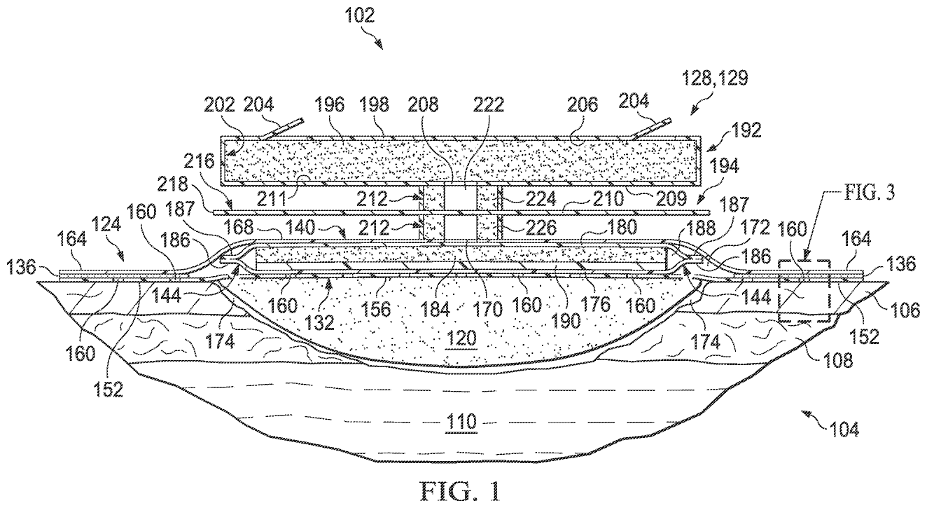

Referring to the drawings, FIG. 1 depicts an embodiment of a system 102 for treating a tissue site 104 of a patient. The tissue site 104 may extend through or otherwise involve an epidermis 106, a dermis 108, and a subcutaneous tissue 110. The tissue site 104 may be a sub-surface tissue site as depicted in FIG. 1 that extends below the surface of the epidermis 106. Further, the tissue site 104 may be a surface tissue site (not shown) that predominantly resides on the surface of the epidermis 106, such as, for example, an incision. The system 102 may provide therapy to, for example, the epidermis 106, the dermis 108, and the subcutaneous tissue 110, regardless of the positioning of the system 102 or the type of tissue site. The system 102 may also be utilized without limitation at other tissue sites.

Further, the tissue site 104 may be the bodily tissue of any human, animal, or other organism, including bone tissue, adipose tissue, muscle tissue, dermal tissue, vascular tissue, connective tissue, cartilage, tendons, ligaments, or any other tissue. Treatment of tissue site 104 may include removal of fluids, e.g., exudate or ascites.

Continuing with FIG. 1, the system 102 may include an optional tissue interface, such as an interface manifold 120. Further, the system 102 may include a dressing 124 and a reduced-pressure source 128. The reduced-pressure source 128 may be or may include a pump module, a pump, or related components described herein. For example, the reduced-pressure source 128 may be referred to as the pump module 129. As indicated above, the interface manifold 120 is an optional component that may be omitted for different types of tissue sites or different types of therapy using reduced pressure, such as, for example, epithelialization. If equipped, the interface manifold 120 may be adapted to be positioned proximate to or adjacent to the tissue site 104, such as, for example, by cutting or otherwise shaping the interface manifold 120 in any suitable manner to fit the tissue site 104. As described below, the interface manifold 120 may be adapted to be positioned in fluid communication with the tissue site 104 to distribute reduced pressure to the tissue site 104. In some embodiments, the interface manifold 120 may be positioned in direct contact with the tissue site 104. The tissue interface or the interface manifold 120 may be formed from any manifold material or flexible bolster material that provides a vacuum space, or treatment space, such as, for example, a porous and permeable foam or foam-like material, a member formed with pathways, a graft, or a gauze. As a more specific, non-limiting example, the interface manifold 120 may be a reticulated, open-cell polyurethane or polyether foam that allows good permeability of fluids while under a reduced pressure. One such foam material is the VAC® GranuFoam® material available from Kinetic Concepts, Inc. (KCI) of San Antonio, Texas. Any material or combination of materials may be used as a manifold material for the interface manifold 120 provided that the manifold material is operable to distribute or collect fluid. For example, herein the term manifold may refer to a substance or structure that is provided to assist in delivering fluids to or removing fluids from a tissue site through a plurality of pores, pathways, or flow channels. The plurality of pores, pathways, or flow channels may be interconnected to improve distribution of fluids provided to and removed from an area around the manifold. Examples of manifolds may include, without limitation, devices that have structural elements arranged to form flow channels, cellular foam, such as open-cell foam, porous tissue collections, and liquids, gels, and foams that include or cure to include flow channels.

A material with a higher or lower density than GranuFoam® material may be desirable for the interface manifold 120 depending on the application. Among the many possible materials, the following may be used: GranuFoam® material, Foamex® technical foam (www.foamex.com), a molded bed of nails structures, a patterned grid material such as those manufactured by Sercol Industrial Fabrics, 3D textiles such as those manufactured by Baltex of Derby, U.K., a gauze, a flexible channel-containing member, a graft, etc. In some instances, ionic silver may be added to the interface manifold 120 by, for example, a micro bonding process. Other substances, such as anti-microbial agents, may be added to the interface manifold 120 as well.

In some embodiments, the interface manifold 120 may comprise a porous, hydrophobic material. The hydrophobic characteristics of the interface manifold 120 may prevent the interface manifold 120 from directly absorbing fluid, such as exudate, from the tissue site 104, but allow the fluid to pass through.

Continuing with FIG. 1, the dressing 124 may be adapted to provide or distribute reduced pressure from the reduced-pressure source 128 to the interface manifold 120, and to store fluid extracted from the tissue site 104 through the interface manifold 120. The dressing 124 may include a base layer 132, an adhesive 136, a sealing member 140, and a fluid management assembly 144. Components of the dressing 124 may be added or removed to suit a particular application.

Referring to FIGS. 1-4B, the base layer 132 may have a periphery 152 surrounding a central portion 156, and a plurality of apertures 160 disposed through the periphery 152 and the central portion 156. The base layer 132 may also have corners 158 and edges 159. The corners 158 and the edges 159 may be part of the periphery 152. One of the edges 159 may meet another of the edges 159 to define one of the corners 158. Further, the base layer 132 may have a border 161 substantially surrounding the central portion 156 and positioned between the central portion 156 and the periphery 152. The border 161 may be free of the apertures 160. The base layer 132 may cover the interface manifold 120 and tissue surrounding the tissue site 104 such that the central portion 156 of the base layer 132 is positioned adjacent to or proximate to the interface manifold 120, and the periphery 152 of the base layer 132 is positioned adjacent to or proximate to tissue surrounding the tissue site 104. In this manner, the periphery 152 of the base layer 132 may surround the interface manifold 120. Further, the apertures 160 in the base layer 132 may be in fluid communication with the interface manifold 120 and tissue surrounding the tissue site 104.

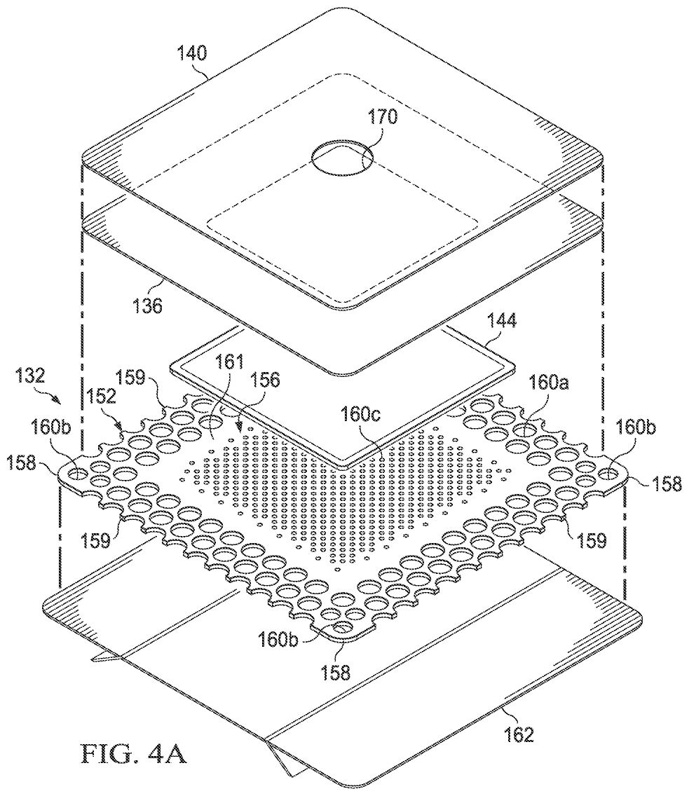

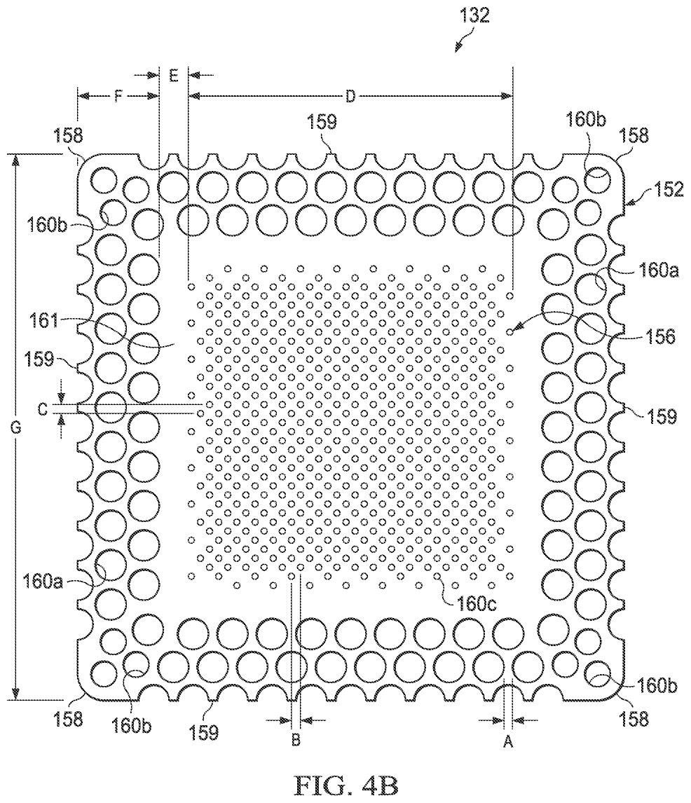

The apertures 160 in the base layer 132 may have any shape, such as, for example, circles, squares, stars, ovals, polygons, slits, complex curves, rectilinear shapes, triangles, or other shapes. The apertures 160 may be formed by cutting, by application of local RF energy, or other suitable techniques for forming an opening. As shown in FIGS. 4A-4B, each of the apertures 160 of the plurality of apertures 160 may be substantially circular in shape, having a diameter and an area. The area of each of the apertures 160 may refer to an open space or open area defining each of the apertures 160. The diameter of each of the apertures 160 may define the area of each of the apertures 160. For example, the area of one of the apertures 160 may be defined by multiplying the square of half the diameter of the aperture 160 by the value 3.14. Thus, the following equation may define the area of one of the apertures 160: Area=3.14*(diameter/2)\hat{ }2. The area of the apertures 160 described in the illustrative embodiments herein may be substantially similar to the area in other embodiments (not shown) for the apertures 160 that may have non-circular shapes. The diameter of each of the apertures 160 may be substantially the same, or each of the diameters may vary depending, for example, on the position of the aperture 160 in the base layer 132. For example, the diameter of the apertures 160 in the periphery 152 of the base layer 132 may be larger than the diameter of the apertures 160 in the central portion 156 of the base layer 132. Further, the diameter of each of the apertures 160 may be between about 1 millimeter to about 50 millimeters. In some embodiments, the diameter of each of the apertures 160 may be between about 1 millimeter to about 20 millimeters. The apertures 160 may have a uniform pattern or may be randomly distributed on the base layer 132. The size and configuration of the apertures 160 may be designed to control the adherence of the dressing 124 to the epidermis 106 as described below.

Referring to FIGS. 4A-4B, in some embodiments, the apertures 160 positioned in the periphery 152 may be apertures 160a, the apertures 160 positioned at the corners 158 of the periphery 152 may be apertures 160b, and the apertures 160 positioned in the central portion 156 may be apertures 160c. The apertures 160a may have a diameter between about 9.8 millimeters to about 10.2 millimeters. The apertures 160b may have a diameter between about 7.75 millimeters to about 8.75 millimeters. The apertures 160c may have a diameter between about 1.8 millimeters to about 2.2 millimeters. The diameter of each of the apertures 160a may be separated from one another by a distance A between about 2.8 millimeters to about 3.2 millimeters. Further, the diameter of at least one of the apertures 160a may be separated from the diameter of at least one of the apertures 160b by the distance A. The diameter of each of the apertures 160b may also be separated from one another by the distance A. A center of one of the apertures 160c may be separated from a center of another of the apertures 160c in a first direction by a distance B between about 2.8 millimeters to about 3.2 millimeters. In a second direction transverse to the first direction, the center of one of the apertures 160c may be separated from the center of another of the apertures 160c by a distance C between about 2.8 millimeters to about 3.2 millimeters. As shown in FIGS. 4A-4B, the distance B and the distance C may be increased for the apertures 160c in the central portion 156 being positioned proximate to or at the border 161 compared to the apertures 160c positioned away from the border 161.

As shown in FIGS. 4A-4B, the central portion 156 of the base layer 132 may be substantially square with each side of the central portion 156 having a length D between about 100 millimeters to about 108 millimeters. In some embodiments, the length D may be between about 106 millimeters to about 108 millimeters. The border 161 of the base layer 132 may have a width E between about 4 millimeters to about 11 millimeters and may substantially surround the central portion 156 and the apertures 160c in the central portion 156. In some embodiments, the width E may be between about 9 millimeters to about 10 millimeters. The periphery 152 of the base layer 132 may have a width F between about 25 millimeters to about 35 millimeters and may substantially surround the border 161 and the central portion 156. In some embodiments, the width F may be between about 26 millimeters to about 28 millimeters. Further, the periphery 152 may have a substantially square exterior with each side of the exterior having a length G between about 154 millimeters to about 200 millimeters. In some embodiments, the length G may be between about 176 millimeters to about 184 millimeters. Although FIGS. 4A-4B depict the central portion 156, the border 161, and the periphery 152 of the base layer 132 as having a substantially square shape, these and other components of the base layer 132 may have any shape to suit a particular application. Further, the dimensions of the base layer 132 as described herein may be increased or decreased, for example, substantially in proportion to one another to suit a particular application. The use of the dimensions in the proportions described above may enhance the cosmetic appearance of a tissue site. For example, these proportions may provide a surface area for the base layer 132, regardless of shape, that is sufficiently smooth to enhance the movement and proliferation of epithelial cells at the tissue site 104, and reduce the likelihood of granulation tissue in-growth into the dressing 124.

The base layer 132 may be a soft, pliable material suitable for providing a fluid seal with the tissue site 104 as described herein. For example, the base layer 132 may comprise a silicone gel, a soft silicone, hydrocolloid, hydrogel, polyurethane gel, polyolefin gel, hydrogenated styrenic copolymer gels, a foamed gel, a soft closed cell foam such as polyurethanes and polyolefins coated with an adhesive described below, polyurethane, polyolefin, or hydrogenated styrenic copolymers. The base layer 132 may have a thickness between about 500 microns (μm) and about 1000 microns (μm). In some embodiments, the base layer 132 has a stiffness between about 5 Shore OO and about 80 Shore OO. The base layer 132 may be comprised of hydrophobic or hydrophilic materials.

In some embodiments (not shown), the base layer 132 may be a hydrophobic-coated material. For example, the base layer 132 may be formed by coating a spaced material, such as, for example, woven, nonwoven, molded, or extruded mesh with a hydrophobic material. The hydrophobic material for the coating may be a soft silicone, for example. In this manner, the adhesive 136 may extend through openings in the spaced material analogous to the apertures 160 described below.

The adhesive 136 may be in fluid communication with the apertures 160 in at least the periphery 152 of the base layer 132. In this manner, the adhesive 136 may be in fluid communication with the tissue surrounding the tissue site 104 through the apertures 160 in the base layer 132. As described below and shown in FIG. 3, the adhesive 136 may extend or be pressed through the plurality of apertures 160 to contact the epidermis 106 for securing the dressing 124 to, for example, the tissue surrounding the tissue site 104. The apertures 160 may provide sufficient contact of the adhesive 136 to the epidermis 106 to secure the dressing 124 about the tissue site 104. However, the configuration of the apertures 160 and the adhesive 136, described below, may permit release and repositioning of the dressing 124 about the tissue site 104.

At least one of the apertures 160a in the periphery 152 of the base layer 132 may be positioned at the edges 159 of the periphery 152 and may have an interior cut open or exposed at the edges 159 that is in fluid communication in a lateral direction with the edges 159. The lateral direction may refer to a direction toward the edges 159 and in the same plane as the base layer 132. As shown in FIGS. 4A-4B, a plurality of the apertures 160a in the periphery 152 may be positioned proximate to or at the edges 159 and in fluid communication in a lateral direction with the edges 159. The apertures 160a positioned proximate to or at the edges 159 may be spaced substantially equidistant around the periphery 152 as shown in FIGS. 4A-4B. However, in some embodiments, the spacing of the apertures 160a proximate to or at the edges 159 may be irregular. The adhesive 136 may be in fluid communication with the edges 159 through the apertures 160a being exposed at the edges 159. In this manner, the apertures 160a at the edges 159 may permit the adhesive 136 to flow around the edges 159 for enhancing the adhesion of the edges 159 around the tissue site 104, for example.

Continuing with FIGS. 4A-4B, the apertures 160b at the corners 158 of the periphery 152 may be smaller than the apertures 160a in other portions of the periphery 152 as described above. For a given geometry of the corners 158, the smaller size of the apertures 160b compared to the apertures 160a may maximize the surface area of the adhesive 136 exposed and in fluid communication through the apertures 160b at the corners 158. For example, as shown in FIGS. 4A-4B, the edges 159 may intersect at substantially a right angle, or about 90 degrees, to define the corners 158. Also as shown, the corners 158 may have a radius of about 10 millimeters. Three of the apertures 160b having a diameter between about 7.75 millimeters to about 8.75 millimeters may be positioned in a triangular configuration at the corners 158 to maximize the exposed surface area for the adhesive 136. The size and number of the apertures 160b in the corners 158 may be adjusted as necessary, depending on the chosen geometry of the corners 158, to maximize the exposed surface area of the adhesive 136 as described above. Further, the apertures 160b at the corners 158 may be fully housed within the base layer 132, substantially precluding fluid communication in a lateral direction exterior to the corners 158. The apertures 160b at the corners 158 being fully housed within the base layer 132 may substantially preclude fluid communication of the adhesive 136 exterior to the corners 158, and may provide improved handling of the dressing 124 during deployment at the tissue site 104. Further, the exterior of the corners 158 being substantially free of the adhesive 136 may increase the flexibility of the corners 158 to enhance comfort.

Similar to the apertures 160b in the corners 158, any of the apertures 160 may be adjusted in size and number to maximize the surface area of the adhesive 136 in fluid communication through the apertures 160 for a particular application or geometry of the base layer 132. For example, in some embodiments (not shown) the apertures 160b, or apertures of another size, may be positioned in the periphery 152 and at the border 161. Similarly, the apertures 160b, or apertures of another size, may be positioned as described above in other locations of the base layer 132 that may have a complex geometry or shape.

The adhesive 136 may be a medically-acceptable adhesive. The adhesive 136 may also be flowable. For example, the adhesive 136 may comprise an acrylic adhesive, rubber adhesive, high-tack silicone adhesive, polyurethane, or other adhesive substance. In some embodiments, the adhesive 136 may be a pressure-sensitive adhesive comprising an acrylic adhesive with coating weight of 15 grams/m² (gsm) to 70 grams/m² (gsm). The adhesive 136 may be a layer having substantially the same shape as the periphery 152 of the base layer 132 as shown in FIG. 4A. In some embodiments, the layer of the adhesive 136 may be continuous or discontinuous. Discontinuities in the adhesive 136 may be provided by apertures (not shown) in the adhesive 136. The apertures in the adhesive 136 may be formed after application of the adhesive 136 or by coating the adhesive 136 in patterns on a carrier layer, such as, for example, a side of the sealing member 140 adapted to face the epidermis 106. Further, the apertures in the adhesive 136 may be sized to control the amount of the adhesive 136 extending through the apertures 160 in the base layer 132 to reach the epidermis 106. The apertures in the adhesive 136 may also be sized to enhance the Moisture Vapor Transfer Rate (MVTR) of the dressing 124, described further below.

Factors that may be utilized to control the adhesion strength of the dressing 124 may include the diameter and number of the apertures 160 in the base layer 132, the thickness of the base layer 132, the thickness and amount of the adhesive 136, and the tackiness of the adhesive 136. An increase in the amount of the adhesive 136 extending through the apertures 160 generally corresponds to an increase in the adhesion strength of the dressing 124. A decrease in the thickness of the base layer 132 generally corresponds to an increase in the amount of adhesive 136 extending through the apertures 160. Thus, the diameter and configuration of the apertures 160, the thickness of the base layer 132, and the amount and tackiness of the adhesive utilized may be varied to provide a desired adhesion strength for the dressing 124. For example, the thickness of the base layer 132 may be about 200 microns, the layer of adhesive 136 may have a thickness of about 30 microns and a tackiness of 2000 grams per 25 centimeter wide strip, and the diameter of the apertures 160a in the base layer 132 may be about 10 millimeters.

In some embodiments, the tackiness of the adhesive 136 may vary in different locations of the base layer 132. For example, in locations of the base layer 132 where the apertures 160 are comparatively large, such as the apertures 160a, the adhesive 136 may have a lower tackiness than other locations of the base layer 132 where the apertures 160 are smaller, such as the apertures 160b and 160c. In this manner, locations of the base layer 132 having larger apertures 160 and lower tackiness adhesive 136 may have an adhesion strength comparable to locations having smaller apertures 160 and higher tackiness adhesive 136.

Clinical studies have shown that the configuration described herein for the base layer 132 and the adhesive 136 may reduce the occurrence of blistering, erythema, and leakage when in use. Such a configuration may provide, for example, increased patient comfort and increased durability of the dressing 124.

Referring to the embodiment of FIG. 4A, a release liner 162 may be attached to or positioned adjacent to the base layer 132 to protect the adhesive 136 prior to application of the dressing 124 to the tissue site 104. Prior to application of the dressing 124 to the tissue site 104, the base layer 132 may be positioned between the sealing member 140 and the release liner 162. Removal of the release liner 162 may expose the base layer 132 and the adhesive 136 for application of the dressing 124 to the tissue site 104. The release liner 162 may also provide stiffness to assist with, for example, deployment of the dressing 124. The release liner 162 may be, for example, a casting paper, a film, or polyethylene. Further, the release liner 162 may be a polyester material such as polyethylene terephthalate (PET), or similar polar semi-crystalline polymer. The use of a polar semi-crystalline polymer for the release liner 162 may substantially preclude wrinkling or other deformation of the dressing 124. For example, the polar semi-crystalline polymer may be highly orientated and resistant to softening, swelling, or other deformation that may occur when brought into contact with components of the dressing 124, or when subjected to temperature or environmental variations, or sterilization. Further, a release agent may be disposed on a side of the release liner 162 that is configured to contact the base layer 132. For example, the release agent may be a silicone coating and may have a release factor suitable to facilitate removal of the release liner 162 by hand and without damaging or deforming the dressing 124. In some embodiments, the release agent may be flourosilicone. In other embodiments, the release liner 162 may be uncoated or otherwise used without a release agent.

Continuing with FIGS. 1-4B, the sealing member 140 has a periphery 164 and a central portion 168. The sealing member 140 may additionally include an aperture 170, as described below. The periphery 164 of the sealing member 140 may be positioned proximate to the periphery 152 of the base layer 132 such that the central portion 168 of the sealing member 140 and the central portion 156 of the base layer 132 define an enclosure 172. The adhesive 136 may be positioned at least between the periphery 164 of the sealing member 140 and the periphery 152 of the base layer 132. The sealing member 140 may cover the tissue site 104 and the interface manifold 120 to provide a fluid seal and a sealed space 174 between the tissue site 104 and the sealing member 140 of the dressing 124. Further, the sealing member 140 may cover other tissue, such as a portion of the epidermis 106, surrounding the tissue site 104 to provide the fluid seal between the sealing member 140 and the tissue site 104. In some embodiments, a portion of the periphery 164 of the sealing member 140 may extend beyond the periphery 152 of the base layer 132 and into direct contact with tissue surrounding the tissue site 104. In other embodiments, the periphery 164 of the sealing member 140, for example, may be positioned in contact with tissue surrounding the tissue site 104 to provide the sealed space 174 without the base layer 132. Thus, the adhesive 136 may also be positioned at least between the periphery 164 of the sealing member 140 and tissue, such as the epidermis 106, surrounding the tissue site 104. The adhesive 136 may be disposed on a surface of the sealing member 140 adapted to face the tissue site 104 and the base layer 132.

The sealing member 140 may be formed from any material that allows for a fluid seal. A fluid seal is a seal adequate to maintain reduced pressure at a desired site given the particular reduced pressure source or system involved. The sealing member 140 may comprise, for example, one or more of the following materials: hydrophilic polyurethane; cellulosics; hydrophilic polyamides; polyvinyl alcohol; polyvinyl pyrrolidone; hydrophilic acrylics; hydrophilic silicone elastomers; an INSPIRE 2301 material from Expopack Advanced Coatings of Wrexham, United Kingdom having, for example, an MVTR (inverted cup technique) of 14400 g/m²/24 hours and a thickness of about 30 microns; a thin, uncoated polymer drape; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; polyurethane (PU); EVA film; co-polyester; silicones; a silicone drape; a 3M Tegaderm® drape; a polyurethane (PU) drape such as one available from Avery Dennison Corporation of Pasadena, California; polyether block polyamide copolymer (PEBAX), for example, from Arkema, France; Expopack 2327; or other appropriate material.

The sealing member 140 may be vapor permeable and liquid impermeable, thereby allowing vapor and inhibiting liquids from exiting the sealed space 174 provided by the dressing 124. In some embodiments, the sealing member 140 may be a flexible, breathable film, membrane, or sheet having a high MVTR of, for example, at least about 300 g/m² per 24 hours. In other embodiments, a low or no vapor transfer drape might be used. The sealing member 140 may comprise a range of medically suitable films having a thickness between about 15 microns (μm) to about 50 microns (μm).

The fluid management assembly 144 may be disposed in the enclosure 172 and may include an absorbent material and optional components that may enhance absorptive efficiency or fluid control and prevention of fluid return to the tissue site 104. In some embodiments, the fluid management assembly 144 may include one or more fluid permeable wicking layers. In other embodiments, one or more of the wicking layers may be omitted and replaced with a perforated film, or the absorbent may be used alone. However, as shown in the examples of FIGS. 1-4B, some embodiments of the fluid management assembly 144 may include a first wicking layer 176, a second wicking layer 180, and an absorbent layer 184. The absorbent layer 184 may be positioned in fluid communication between the first wicking layer 176 and the second wicking layer 180. The first wicking layer 176 may have a grain structure (not shown) adapted to wick fluid along a surface of the first wicking layer 176. Similarly, the second wicking layer 180 may have a grain structure (not shown) adapted to wick fluid along a surface of the second wicking layer 180. For example, the first wicking layer 176 and the second wicking layer 180 may wick or otherwise transport fluid in a lateral direction along the surfaces of the first wicking layer 176 and the second wicking layer 180, respectively. The surfaces of the first wicking layer 176 and the second wicking layer 180 may be normal relative to the thickness of each of the first wicking layer 176 and the second wicking layer 180. The wicking of fluid along the first wicking layer 176 and the second wicking layer 180 may enhance the distribution of the fluid over a surface area of the absorbent layer 184 that may increase absorbent efficiency and resist fluid blockages. Fluid blockages may be caused by, for example, fluid pooling in a particular location in the absorbent layer 184 rather than being distributed more uniformly across the absorbent layer 184. The laminate combination of the first wicking layer 176, the second wicking layer 180, and the absorbent layer 184 may be adapted as described above to maintain an open structure, resistant to blockage, capable of maintaining fluid communication with, for example, the tissue site 104.

A peripheral portion 186 of the first wicking layer 176 may be coupled to a peripheral portion 187 of the second wicking layer 180 to define a wicking layer enclosure 188 between the first wicking layer 176 and the second wicking layer 180. In some exemplary embodiments, the wicking layer enclosure 188 may surround or otherwise encapsulate the absorbent layer 184 between the first wicking layer 176 and the second wicking layer 180.

In some embodiments, the absorbent layer 184 may be a hydrophilic material adapted to absorb fluid from, for example, the tissue site 104. Materials suitable for the absorbent layer 184 may include Luquafleece® material, Texsus FP2326, BASF 402C, Technical Absorbents 2317 available from Technical Absorbents (www.techabsorbents.com), sodium polyacrylate super absorbers, cellulosics (carboxy methyl cellulose and salts such as sodium CMC), or alginates. Materials suitable for the first wicking layer 176 and the second wicking layer 180 may include any material having a grain structure capable of wicking fluid as described herein, such as, for example, Libeltex TDL2 80 gsm.

The fluid management assembly 144 may be a pre-laminated structure manufactured at a single location or individual layers of material stacked upon one another as described above. Individual layers of the fluid management assembly 144 may be bonded or otherwise secured to one another without adversely affecting fluid management by, for example, utilizing a solvent or non-solvent adhesive, or by thermal welding. Further, the fluid management assembly 144 may be coupled to the border 161 of the base layer 132 in any suitable manner, such as, for example, by a weld or an adhesive. The border 161 being free of the apertures 160 as described above may provide a flexible barrier between the fluid management assembly 144 and the tissue site 104 for enhancing comfort.

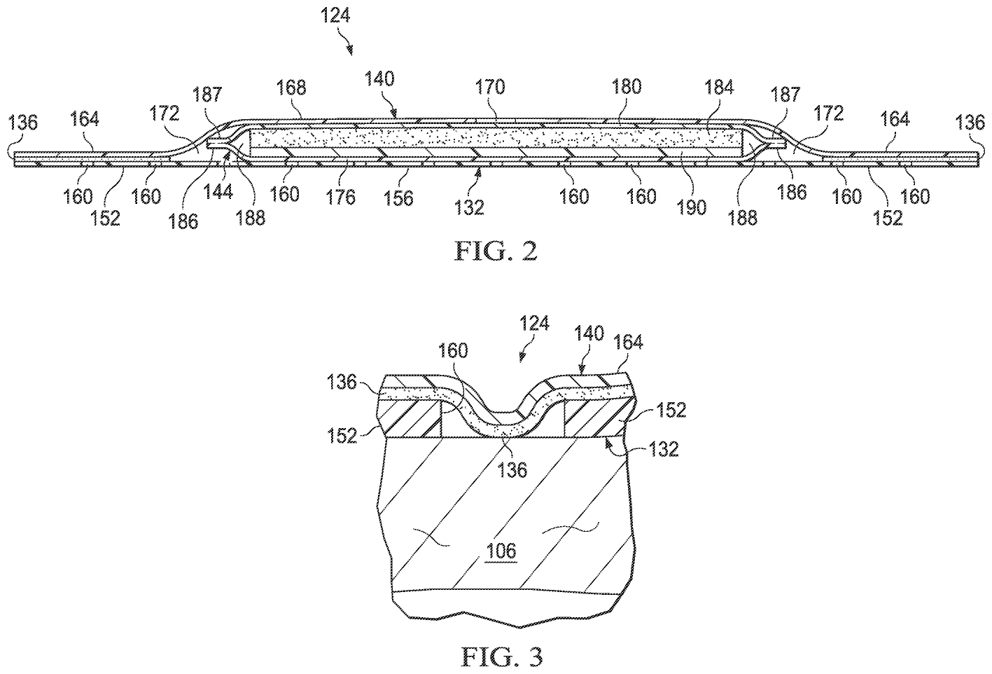

In some embodiments, the enclosure 172 defined by the base layer 132 and the sealing member 140 may include an anti-microbial layer 190. The addition of the anti-microbial layer 190 may reduce the probability of excessive bacterial growth within the dressing 124 to permit the dressing 124 to remain in place for an extended period. The anti-microbial layer 190 may be, for example, an additional layer included as a part of the fluid management assembly 144 as depicted in FIGS. 1 and 2, or a coating of an anti-microbial agent disposed in any suitable location within the dressing 124. The anti-microbial layer 190 may be comprised of elemental silver or similar compound, for example. In some embodiments, the anti-microbial agent may be formulated in any suitable manner into other components of the dressing 124.

Referring to FIGS. 1, 5A, and 5B, the reduced-pressure source 128 may be the pump module 129 which may be positioned proximate to the sealing member 140 and in fluid communication with the dressing 124 through the aperture 170 in the sealing member 140. The pump module 129 may include a pump 192 and a pump actuation assembly 194.

The pump 192 may include a biasing element 196 and a pump housing 198. The biasing element 196 may be configured to generate a variable volume within the pump housing 198 when the pump 192 is activated. The biasing element 196 may be in a compressed state prior to activation of the pump 192 as shown in FIG. 5A. In the compressed state, the biasing element 196 may have a first volume 195. After activation of the pump 192, the biasing element 196 may expand to an expanded state as shown in FIG. 5B. In the expanded state, the biasing element 196 may have a second volume 197 that may be greater than the first volume 195. Further, the expanded state and the second volume 197 may be the normal resting state for the biasing element 196 and the at rest volume for the pump housing 198 prior to placement or compression of the biasing element 196 into the compressed state. As the biasing element 196 moves or expands from the compressed state to the expanded state to return to its normal resting position, a variable or gradual increase in volume may occur in the pump housing 198. Once the pump 192 is activated and the pump housing 198 begins to increase in volume, a force, such as a vacuum or reduced pressure, may be exerted upon the dressing 124. In some embodiments, the pump 192 may be able to maintain a reduced pressure at the dressing for several days.

As used herein, “reduced pressure” generally refers to a pressure less than the ambient pressure at a tissue site being subjected to treatment. Typically, this reduced pressure will be less than the atmospheric pressure. The reduced pressure may also be less than a hydrostatic pressure at a tissue site. Unless otherwise indicated, values of pressure stated herein are gauge pressures. While the amount and nature of reduced pressure applied to a tissue site will typically vary according to the application, the reduced pressure will typically be between −5 mm Hg and −500 mm Hg, and more typically in a therapeutic range between-100 mm Hg and −200 mm Hg.

The reduced pressure delivered may be constant or varied (patterned or random), and may be delivered continuously or intermittently. Although the terms “vacuum” and “negative pressure” may be used to describe the pressure applied to the tissue site, the actual pressure applied to the tissue site may be more than the pressure normally associated with a complete vacuum. Consistent with the use herein, an increase in reduced pressure or vacuum pressure typically refers to a relative reduction in absolute pressure. An increase in reduced pressure corresponds to a reduction in pressure (more negative relative to ambient pressure) and a decrease in reduced pressure corresponds to an increase in pressure (less negative relative to ambient pressure).

In some embodiments, the biasing element 196 may be configured to store exudate from the tissue site 104. The biasing element 196 may be configured to store certain amounts of exudate depending on the volume of the biasing element 196. For example, a biasing element 196 of 8 cm by 3 cm by 1.5 cm may be configured to hold about 36 mL of fluid. A biasing element 196 of 15 cm by 8 cm by 1.5 cm may be configured to hold about 180 mL of fluid. A biasing element 196 of 15 cm by 8 cm by 0.5 cm may be configured to hold about 60 mL of fluid. A biasing element 196 of 15 cm by 15 cm by 1.5 cm may be configured to hold about 338 mL of fluid. A biasing element 196 of 15 cm by 3 cm by 1 cm may be configured to hold about 45 mL of fluid. In some embodiments, there may be an additional absorbent material (not shown) included within the pump housing 198 of the pump 192 in any suitable manner, such as, for example an additional layer or pouch of absorbent, or absorbent particles impregnated within the biasing element 196. The absorbent material may be configured to absorb fluid from the tissue site 104 that is drawn into the pump housing 198 by the biasing element 196. The pump housing 198 may be configured to expand with the expansion of the biasing element 196 and the absorbent material. Materials suitable for the absorbent material may include Luquafleece® material, Texsus FP2326, BASF 402C, Technical Absorbents 2317 available from Technical Absorbents (www.techabsorbents.com), sodium polyacrylate super absorbers, cellulosics (carboxy methyl cellulose and salts such as sodium CMC), or alginates.

In some embodiments, the biasing element 196 may be foam. The foam may be configured to create a relatively constant reduced-pressure at the dressing 124 while the pump 192 is moving from a compressed state to an expanded state. The foam may have a plurality of interconnected flow channels and may be, for example, cellular foam, open-cell foam, reticulated foam, porous tissue collections, and other porous materials that generally include pores, edges, and/or walls adapted to form interconnected fluid pathways. In some illustrative embodiments, the foam may be a porous foam material having interconnected cells or pores adapted to uniformly (or quasi-uniformly) distribute fluid throughout the foam. The foam material may be either hydrophobic or hydrophilic. In one non-limiting example, the foam may be an open-cell, reticulated polyurethane foam such as V.A.C.® GRANUFOAM™ dressing available from Kinetic Concepts, Inc. of San Antonio, Texas.

Foam materials may have an elastic modulus, which may also be referred to as a foam modulus. Generally, the elastic modulus of a material may measure the resistance of the material to elastic deformation under a load. The elastic modulus of a material may be defined as the slope of a stress-strain curve in the elastic deformation region of the curve. The elastic deformation region of a stress-strain curve represents that portion of the curve where a deformation of a material due to an applied load is elastic, that is, not permanent. If the load is removed, the material may return to its preloaded state. Stiffer materials may have a higher elastic modulus, and more compliant materials may have a lower elastic modulus. Generally, references to the elastic modulus of a material refers to a material under tension.

For some materials under compression, the elastic modulus can be compared between materials by comparing the compression force deflection (CFD) of the materials. Typically, CFD is determined experimentally by compressing a sample of a material until the sample is reduced by about 25% of its uncompressed thickness. The load applied to reach the 25% compression of the sample is then divided by the area of the sample over which the load is applied to arrive at the CFD. The CFD can also be measured by compressing a sample of a material to about 50% of the sample's uncompressed size. The CFD of a foam material can be a function of compression level, polymer stiffness, cell structure, foam density, and cell pore size. In some illustrative examples, the foam may have a CFD at 25% compression of about 10 kPA to 20 kPA. The foam in this example may compress to about 25% of its uncompressed size if a load of about 10 kPA to 20 kPA is applied to the foam.

Furthermore, CFD can represent the tendency of a foam to return to its uncompressed state if a load is applied to compress the foam. For example, a foam having a CFD of about 10 kPA to 20 kPA may exert about 10 kPA to 20 kPA in reaction to 25% compression. The CFD of the foam may represent the ability of the foam to bias the pump housing 198, or a component thereof, toward an expanded position. For example, if the foam is compressed to 25% of its original size, the foam may exert a reactive force, such as a spring force, that opposes the applied force over the area of the foam to which the force is applied. The reactive force may be proportional to the amount the foam is compressed.

In some illustrative examples, the pump housing 198 may be or may include a film that defines at least a portion of a sealed chamber 202 that encases the biasing element 196. The film of the pump housing 198 may be formed of an elastic or resilient material to permit the pump housing 198 to compress and expand with the compression and expansion of the biasing element 196. In some embodiments, the pump housing 198 may include openings, check valves, or exhaust ports configured to enable fluid communication between the sealed chamber 202 and/or the biasing element 196 and the dressing 124 or the ambient environment. In some embodiments, the pump housing 198 may include more than one layer of film.

In some embodiments, the pump housing 198 may include one or more check valves 204. Exemplary check valves 204 may include ball check valves, diaphragm check valves, flap-style check valves, swing check valves, stop-check valves, duckbill valves, pneumatic non-return valves, or other one-way valves configured to automatically permit fluid flow in a single direction and to prevent fluid flow in any other direction. FIGS. 1 and 5B illustrate a flap or swing embodiment of the check valves 204 in an open state configured to permit fluid to exhaust or vent out of the sealed chamber 202 of the pump housing 198 to the external ambient environment as explained further below. FIG. 5A illustrates the check valves 204 in a closed state prior to activation of the pump 192 and while the sealed chamber 202 of the pump housing 198 is under reduced-pressure, for example, as a result of the biasing element 196 being under some amount of compression. The presence of reduced-pressure within the sealed chamber 202 of the pump housing 198 may assist in drawing or maintaining some embodiments of the check valves 204 in the closed state. Check valves 204 may be located through the pump housing 198 proximate to a first surface 206 of the biasing element 196. The check valves 204 may be configured to permit fluid communication in a direction from the sealed chamber 202 and/or the biasing element 196 toward the ambient environment and to preclude fluid communication in the opposite direction from the ambient environment toward the sealed chamber 202 and/or the biasing element 196.

In some embodiments, the check valves 204 may allow for re-priming of the pump 192 as desired or, for example, once the biasing element 196 has reached the expanded state. A user can pinch the pump 192 to move the biasing element 196 from the expanded state to a re-compressed state. The check valves 204 may allow fluid to exhaust, vent, or otherwise escape from the pump housing 198 in order to re-compress the biasing element 196.

There may be an opening 208 in a base 209 of the pump housing 198 proximate to a second surface 211 of the biasing element 196. The opening 208 may be configured to enable fluid communication among the sealed chamber 202 and/or the biasing element 196 and the dressing 124. In some embodiments, there may be a check valve (not shown) proximate to or in fluid communication with the opening 208 that is analogous to the check valves 204, but having an opposite orientation. For example, the check valve proximate to the opening 208 may be configured to permit fluid communication in a direction from the dressing 124 toward the sealed chamber 202 and/or the biasing element 196 and to prevent fluid communication in the opposite direction from the sealed chamber 202 and/or the biasing element 196 toward the dressing 124.

In some embodiments, the pump housing 198 may include at least one exhaust port configured to enable fluid communication from the sealed chamber 202 to the ambient environment. The at least one exhaust port can be located through the pump housing 198 proximate to the first surface 206 of the biasing element 196. The at least one exhaust port may be included in embodiments with or without the check valves 204.

In some embodiments, there may be a hydrophobic filter disposed between the biasing element 196 and the pump housing 198. For example, the hydrophobic filter may be a liquid impermeable and gas permeable layer configured to cover the opening 208 in the pump housing 198 for blocking liquid communication into the pump housing 198. In embodiments with the hydrophobic filter, the biasing element 196 may be configured to generate a reduced pressure at the dressing 124, but may not be configured to store exudate from the tissue site 104.

The pump housing 198 may be formed from any material that allows for a fluid seal. A fluid seal is a seal adequate to maintain reduced pressure at a desired site given the particular reduced pressure source or system involved. The pump housing 198 may comprise, for example, one or more of the following materials: hydrophilic polyurethane; cellulosics; hydrophilic polyamides; polyvinyl alcohol; polyvinyl pyrrolidone; hydrophilic acrylics; hydrophilic silicone elastomers; an INSPIRE 2301 material from Expopack Advanced Coatings of Wrexham, United Kingdom having, for example, an MVTR (inverted cup technique) of 14400 g/m²/24 hours and a thickness of about 30 microns; a thin, uncoated polymer drape; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; polyurethane (PU); EVA film; co-polyester; silicones; a silicone drape; a 3M Tegaderm® drape; a polyurethane (PU) drape such as one available from Avery Dennison Corporation of Pasadena, California; polyether block polyamide copolymer (PEBAX), for example, from Arkema, France; Expopack 2327; or other appropriate material.

The pump housing 198 may be vapor permeable and liquid impermeable, thereby allowing vapor and inhibiting liquids from exiting the sealed chamber 202. In some embodiments, the pump housing 198 may be a flexible, breathable film, membrane, or sheet having a high MVTR of, for example, at least about 300 g/m² per 24 hours. In other embodiments, a low or no vapor transfer drape might be used. The pump housing 198 may comprise a range of medically suitable films having a thickness between about 15 microns (μm) to about 50 microns (μm). In other embodiments, the pump housing 198 may be a non-breathable film, membrane, or sheet that may be substantially vapor and liquid impermeable.

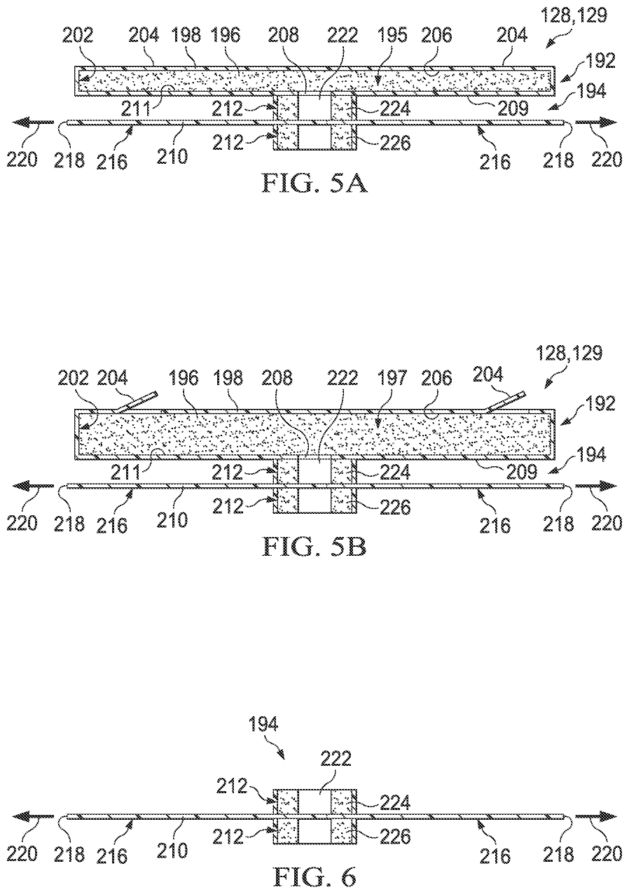

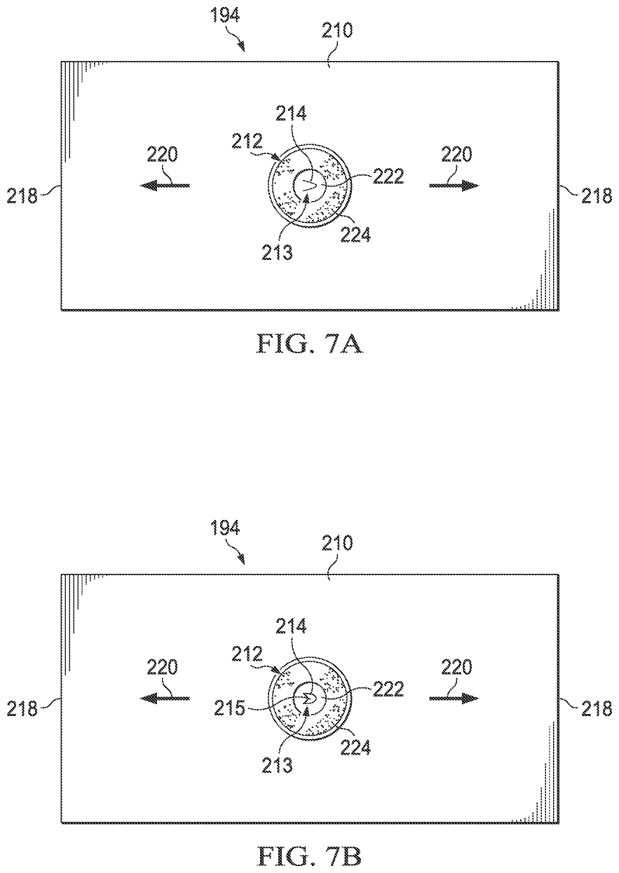

Referring to FIGS. 1, 5A-5B, 6, and 7A-7B, the pump module 129 may include the pump actuation assembly 194. The pump actuation assembly 194 may be positioned in fluid communication between the pump 192 and the dressing 124. For example, a port 212 may be defined through the pump actuation assembly 194 and configured to enable fluid communication between the dressing 124 and the pump 192. Further, in some illustrative examples, the pump actuation assembly 194 may include a sacrificial seal 213 and an actuation device 216 configured to activate the pump 192 when the sacrificial seal 213 is broken. For example, the sacrificial seal 213 may include or may be a film layer 210 including a zone of weakness 214. In some illustrative examples, the actuation device 216 may include at least one pull tab 218 configured to stretch the film layer 210 to form an opening 215, shown in FIG. 7B, at the zone of weakness 214 and to activate the pump 192 when pulled. In some illustrative example embodiments, the at least one pull tab 218 may be formed integrally with or as part of the film layer 210 and may be an extension or extended portion of the film layer 210. In other embodiments, the at least one pull tab 218 may be a separate gripping structure coupled to the film layer 210.

The zone of weakness 214 may be an area of the film layer 210 that is configured to break when a force such as force 220 is exerted on the film layer 210. The zone of weakness 214 may be created in the film layer 210 by a controlled depth cut configured to fail or perforate when exposed to tension. In some embodiments, the film layer 210 may include an area around the zone of weakness 214 that may include a portion or another material with a higher tensile strength than the substrate material from which the film layer 210 is formed. This area may ensure that the film layer 210 fails or perforates only at the zone of weakness 214 and not at any other location on the film layer 210. The actuation device 216 may be configured to allow the user to exert the force 220 with the pull tabs 218. For example, the user may pull the pull tabs 218 laterally away from one another and away from the zone of weakness 214 in the direction of the force 220. The force 220 may stretch the film layer 210 to form the opening 215 through the sacrificial seal 213 at the zone of weakness 214, positioning the sacrificial seal 213 in an open or activated state as shown in FIG. 7B. Prior to being activated, the sacrificial seal 213 is closed or in a sealed state as shown in FIG. 7A. When the opening 215 is formed in the film layer 210 and the sacrificial seal 213 is in the activated state, the pump 192 may also be activated. When the pump 192 is activated, fluid from the dressing 124 may flow through the opening 215 to the pump 192.

The film layer 210 may be configured to maintain the compressed state of the biasing element 196 prior to forming the opening 215 at the zone of weakness 214. When the opening 215 is created in the film layer 210 at the zone of weakness 214, the pump 192 may be activated and may generate reduced pressure at the dressing 124. The film layer 210 may be formed from any material that allows for a fluid seal. The material must be strong enough to maintain the fluid seal even after the zone of weakness 214 is created in the film layer 210. For example, the film layer 210 may comprise, for example, one or more of the following materials: hydrophilic polyurethane; cellulosics; hydrophilic polyamides; polyvinyl alcohol; polyvinyl pyrrolidone; hydrophilic acrylics; hydrophilic silicone elastomers; an INSPIRE 2301 material from Expopack Advanced Coatings of Wrexham, United Kingdom having, for example, an MVTR (inverted cup technique) of 14400 g/m²/24 hours and a thickness of about 30 microns; a thin, uncoated polymer drape; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; polyurethane (PU); EVA film; co-polyester; silicones; a silicone drape; a 3M Tegaderm® drape; a polyurethane (PU) drape such as one available from Avery Dennison Corporation of Pasadena, California; polyether block polyamide copolymer (PEBAX), for example, from Arkema, France; Expopack 2327; or other appropriate material.

The pump actuation assembly 194 may be configured to enable fluid communication between the pump 192 and the dressing 124 through the port 212. The sacrificial seal 213 may be positioned in the port 212 to prevent fluid communication through the port 212 until the sacrificial seal 213 is activated to form the opening 215. In some illustrative examples, the port 212 may be comprised of closed cell foam. For example, the port 212 may be a sealing structure, donut, or a ring having a passage 222 defined therethrough. In some embodiments, the port 212 may comprise two rings or portions of closed cell foam. A first portion of the port 212 may be a pump mounting portion 224 of the pump actuation assembly 194 that may be configured to be positioned between the pump 192 and the film layer 210. The pump mounting portion 224 of the pump actuation assembly 194 may be coupled to the pump 192 by adhesively bonding, welding, or attaching in any other suitable manner. A second portion of the port 212 may be an external mounting portion 226 of the pump actuation assembly 194 that may be configured to be positioned between the film layer 210 and the dressing 124, opposite from the pump mounting portion 224. The external mounting portion 226 of the port 212 may be coupled to the dressing 124 by adhesively bonding, welding, or attaching in any other suitable manner. In some examples, an exterior surface of the external mounting portion 226 may carry a releasable adhesive.

The passage 222 of the pump mounting portion 224 of the port 212 may be aligned with the opening 208 of the pump housing 198. The passage 222 of the external mounting portion 226 may be aligned with the aperture 170 in the dressing 124 or otherwise positioned in fluid communication with the dressing 124. The film layer 210 may be positioned in the port 212 of the pump actuation assembly 194. A portion of the sacrificial seal 213, such as the zone of weakness 214 may be located on an area of the film layer 210 in the passage 222 of the port 212 and between the pump mounting portion 224 and the external mounting portion 226. When the film layer 210 is broken at the zone of weakness 214, the opening 215 in the film layer 210 can provide fluid communication between the dressing 124 and the pump 192 through the passage 222 of the port 212.

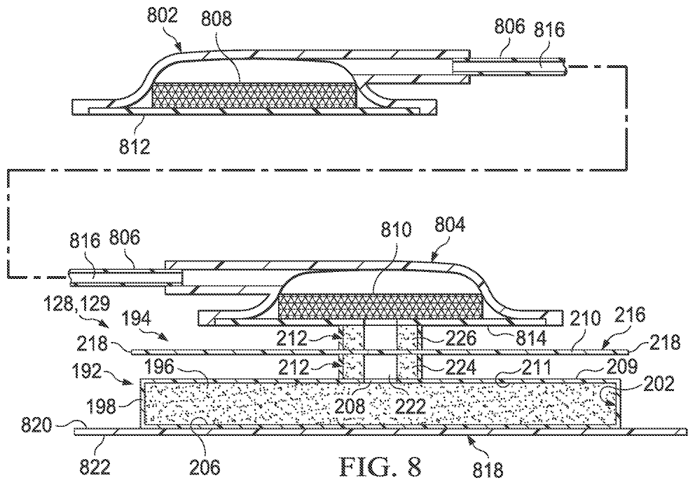

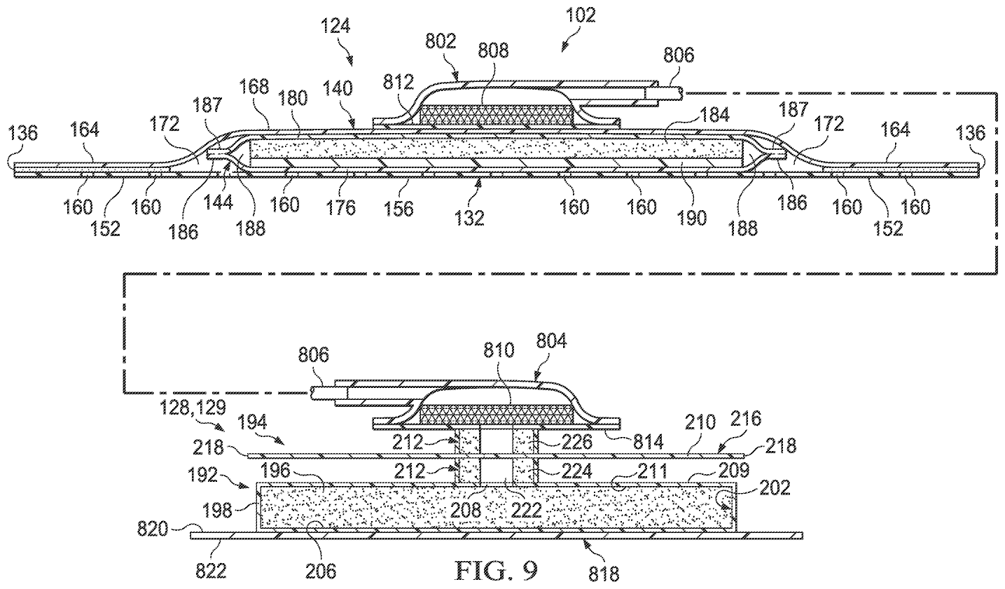

In some illustrative examples, a portion of the pump actuation assembly 194, such as the port 212, may be coupled directly to both the pump 192 and the dressing 124 as shown in FIG. 1. Further, the pump 192 may carry the pump actuation assembly 194, or the pump 192 and the pump actuation assembly 194 may otherwise be coupled together as an assembled unit, permitting a caregiver conveniently couple both the pump 192 and the pump actuation assembly 194 to the dressing 124. In other illustrative examples, a conduit may be coupled in fluid communication between the pump 192 and the dressing 124 with the pump actuation assembly 194 positioned at the pump 192, at the dressing 124, or any other suitable location in fluid communication between the pump 192 and the dressing 124. As described further below in reference to FIGS. 8 and 9, in some examples, a portion of the pump actuation assembly 194, such as the port 212, may be configured to be coupled directly to the pump 192 with a conduit being coupled in fluid in fluid communication between the port 212 and the dressing 124.

In some embodiments not pictured herein, the pump actuation assembly 194 may not include the port 212. The film layer 210 may be coupled to and between the pump 192 and the dressing 124 by adhesively bonding, welding, or attaching in any other suitable manner. When the zone of weakness 214 is broken, the opening 215 may align with the opening 208 of the pump housing 198 and the aperture 170 of the dressing 124 in order to provide fluid communication between the pump 192 and the dressing 124.

Further, the illustrative example embodiments of the port 212 are depicted in the figures with an enlarged thickness to enhance visualization such that the various components of the port 212 are not depicted to scale. In other examples, at least a portion of the base 209 of the pump housing 198 may be positioned adjacent to the film layer 210, and at least a portion of the film layer 210 may be positioned adjacent to the dressing 124.

Referring to FIGS. 8 and 9, some illustrative example embodiments of the system 102 may include the dressing 124, a first conduit interface 802, a second conduit interface 804, a conduit 806, the pump 192, and the pump actuation assembly 194. These embodiments may be used when it is desirable for the pump module 129 to be located separately from the dressing 124. The first conduit interface 802 may be positioned proximate to the sealing member 140 and in fluid communication with the dressing 124 through the aperture 170 in the sealing member 140 to provide reduced pressure from the pump module 129 to the dressing 124. Specifically, the first conduit interface 802 may be positioned in fluid communication with the enclosure 172 of the dressing 124. The first conduit interface 802 may also be positioned in fluid communication with the optional interface manifold 120, shown in FIG. 1.

The second conduit interface 804 may be positioned proximate to the port 212 of the pump actuation assembly 194. The second conduit interface 804 may be in fluid communication with the pump 192 through the passage 222 of the port 212 and the opening 208 in the pump housing 198. The second conduit interface 804 may provide reduced pressure from the pump module 129 to the first conduit interface 802 and to the dressing 124.

The first conduit interface 802 and the second conduit interface 804 may comprise a medical-grade, soft polymer or other pliable material. As non-limiting examples, the first conduit interface 802 and the second conduit interface 804 may be formed from polyurethane, polyethylene, polyvinyl chloride (PVC), fluorosilicone, or ethylene-propylene, etc. In some illustrative, non-limiting embodiments, the first conduit interface 802 and the second conduit interface 804 may be molded from DEHP-free PVC. The first conduit interface 802 and the second conduit interface 804 may be formed in any suitable manner such as by molding, casting, machining, or extruding. Further, the first conduit interface 802 and the second conduit interface 804 may be formed as an integral unit or as individual components and may be coupled respectively to the dressing 124 and the port 212 by, for example, adhesive or welding.

In some embodiments, the first conduit interface 802 and the second conduit interface 804 may be formed of an absorbent material having absorbent and evaporative properties. The absorbent material may be vapor permeable and liquid impermeable, thereby being configured to permit vapor to be absorbed into and evaporated from the material through permeation while inhibiting permeation of liquids. The absorbent material may be, for example, a hydrophilic polymer such as a hydrophilic polyurethane. Although the term hydrophilic polymer may be used in the illustrative embodiments that follow, any absorbent material having the properties described herein may be suitable for use in the system 102. Further, the absorbent material or hydrophilic polymer may be suitable for use in various components of the system 102 as described herein.

The use of such a hydrophilic polymer for the first conduit interface 802 and the second conduit interface 804 may permit liquids in the first conduit interface 802 and the second conduit interface 804 to evaporate, or otherwise dissipate, during operation. For example, the hydrophilic polymer may allow the liquid to permeate or pass through the first conduit interface 802 and the second conduit interface 804 as vapor, in a gaseous phase, and evaporate into the atmosphere external to the first conduit interface 802 and the second conduit interface 804. Such liquids may be, for example, condensate or other liquids. Condensate may form, for example, as a result of a decrease in temperature within the first conduit interface 802 or the second conduit interface 804, or other components of the system 102, relative to the temperature at the tissue site 104. Removal or dissipation of liquids from the first conduit interface 802 and the second conduit interface 804 may increase visual appeal and prevent odor. Further, such removal of liquids may also increase efficiency and reliability by reducing blockages and other interference with the components of the system 102.

In some embodiments, the absorbent material or hydrophilic polymer may have an absorbent capacity in a saturated state that is substantially equivalent to the mass of the hydrophilic polymer in an unsaturated state. The hydrophilic polymer may be fully saturated with vapor in the saturated state and substantially free of vapor in the unsaturated state. In both the saturated state and the unsaturated state, the hydrophilic polymer may retain substantially the same physical, mechanical, and structural properties. For example, the hydrophilic polymer may have a hardness in the unsaturated state that is substantially the same as a hardness of the hydrophilic polymer in the saturated state. The hydrophilic polymer and the components of the system 102 incorporating the hydrophilic polymer may also have a size that is substantially the same in both the unsaturated state and the saturated state. Further, the hydrophilic polymer may remain dry, cool to the touch, and pneumatically sealed in the saturated state and the unsaturated state. The hydrophilic polymer may also remain substantially the same color in the saturated state and the unsaturated state. In this manner, this hydrophilic polymer may retain sufficient strength and other physical properties to remain suitable for use in the system 102. An example of such a hydrophilic polymer is offered under the trade name Techophilic HP-93A-100, available from The Lubrizol Corporation of Wickliffe, Ohio, United States. Techophilic HP-93A-100 is an absorbent hydrophilic thermoplastic polyurethane capable of absorbing 100% of the unsaturated mass of the polyurethane in water and having a durometer or Shore Hardness of about 83 Shore A.

The first conduit interface 802 may optionally carry an odor filter 808 and the second conduit interface 804 may optionally carry an odor filter 810 adapted to substantially preclude the passage of odors from the tissue site 104 out of the sealed space 174. Further, the first conduit interface 802 may optionally carry a gas permeable and liquid impermeable hydrophobic filter 812 adapted to substantially preclude the passage of liquids out of the sealed space 174. Similarly, the second conduit interface 804 may optionally carry a gas permeable and liquid impermeable hydrophobic filter 814 adapted to substantially preclude the passage of liquids out of the conduit 806 or into the pump 192. The odor filter 808 and the hydrophobic filter 812 may be disposed in the first conduit interface 802 and the odor filter 810 and the hydrophobic filter 814 may be disposed in the second conduit interface 804 or other suitable location such that fluid communication between the pump module 129 and the dressing 124 is provided through the odor filter 808, the odor filter 810, the hydrophobic filter 812, and the hydrophobic filter 814. In some embodiments, the odor filter 808 and the hydrophobic filter 812 may be secured within the first conduit interface 802 in any suitable manner, such as by adhesive or welding. In some embodiments, the odor filter 810 and the hydrophobic filter 814 may be secured within the second conduit interface 804 in any suitable manner, such as by adhesive or welding. In other embodiments, the odor filter 808 and the hydrophobic filter 812 may be positioned in any exit location in the dressing 124 that is in fluid communication with the atmosphere. The odor filter 808 may also be positioned in any suitable location in the system 102 that is in fluid communication with the tissue site 104.

The odor filter 808 and the odor filter 810 may be comprised of a carbon material in the form of a layer or particulate. For example, the odor filter 808 and the odor filter 810 may comprise a woven carbon cloth filter such as those manufactured by Chemviron Carbon, Ltd. of Lancashire, United Kingdom (www.chemvironcarbon.com). The hydrophobic filter 812 and the hydrophobic filter 814 may be comprised of a material that is liquid impermeable and vapor permeable. For example, the hydrophobic filter 812 and the hydrophobic filter 814 may comprise a material manufactured under the designation MMT-314 by W.L. Gore & Associates, Inc. of Newark, Delaware, United States, or similar materials. The hydrophobic filter 812 and the hydrophobic filter 814 may be provided in the form of a membrane or layer.

The conduit 806 has an internal lumen 816 and may be coupled in fluid communication between the pump module 129 and the dressing 124. The internal lumen 816 may have an internal diameter between about 0.5 millimeters to about 3.0 millimeters. More specifically, the internal diameter of the internal lumen 816 may be between about 1 millimeter to about 2 millimeters. The first conduit interface 802 may be coupled in fluid communication with the dressing 124 and the second conduit interface 804 may be coupled in fluid communication with the pump module 129. The first conduit interface 802 and the second conduit interface 804 may be adapted to connect to the conduit 806 to provide fluid communication between the dressing 124 and the reduced-pressure source 128. The first conduit interface 802 and the second conduit interface 804 may be fluidly coupled to the conduit 806 in any suitable manner, such as, for example, by an adhesive, solvent or non-solvent bonding, welding, or interference fit. The aperture 170 in the sealing member 140 may provide fluid communication between the dressing 124 and the first conduit interface 802. Specifically, the first conduit interface 802 may be in fluid communication with the enclosure 172 or the sealed space 174 through the aperture 170 in the sealing member 140. The passage 222 of the port 212 may provide fluid communication between the pump module 129 and the second conduit interface 804.

In some embodiments, the conduit 806 may be inserted into the dressing 124 through the aperture 170 in the sealing member 140 to provide fluid communication with the pump module 129 without use of the first conduit interface 802. The conduit 806 may further be inserted directly into the passage 222 of the port 212 of the pump actuation assembly 194 to provide fluid communication between the dressing 124 and the pump module 129 without the second conduit interface 804. The conduit 806 may be, for example, a flexible polymer tube.

In some embodiments, the conduit 806 may be formed of an absorbent material or a hydrophilic polymer as described above for the first conduit interface 802 and the second conduit interface 804. In this manner, the conduit 806 may permit liquids in the conduit 806 to evaporate, or otherwise dissipate, as described above for the first conduit interface 802 and the second conduit interface 804. Further, a wall of the conduit 806 defining the internal lumen 816 may be extruded from the hydrophilic polymer. The conduit 806 may be less than about 1 meter in length, but may have any length to suit a particular application. More specifically, a length of about 1 foot or 304.8 millimeters may provide enough absorbent and evaporative surface area to suit many applications, and may provide a cost savings compared to longer lengths. If an application requires additional length for the conduit 806, the absorbent hydrophilic polymer may be coupled in fluid communication with a length of conduit formed of a non-absorbent hydrophobic polymer to provide additional cost savings.

Referring to FIGS. 8 and 9, some embodiments of the system 102 may require the pump module 129 to be located at a different location on the user's body than the dressing 124. The pump 192 may be coupled to the pump actuation assembly 194 and a contact layer 818 configured to be in contact with a user. The first surface 206 of the biasing element 196 may be proximate to the contact layer 818 and the second surface 211 of the biasing element 196 may be proximate to the pump actuation assembly 194. The pump mounting portion 224 of the pump actuation assembly 194 may be coupled to the pump 192 so that the passage 222 of the port 212 may be aligned with the opening 208 of the pump housing 198. The external mounting portion 226 of the port 212 may be coupled to the second conduit interface 804. The second conduit interface 804 may be in fluid communication with the first conduit interface 802 and the pump 192 when the opening 215 is created at the zone of weakness 214 in the film layer 210 of the pump actuation assembly 194.

The contact layer 818 may have a first surface 820 coupled to the pump 192 and a second surface 822 configured to be coupled to a user. The second surface 822 may include an adhesive covered by a release liner such that when the release liner is removed, the adhesive can be exposed in order to couple the pump module 129 to the user. The pump housing 198 may have exhaust ports or check valves located proximate to the second surface 211 of the biasing element 196 or in another location that is not covered by the contact layer 818.

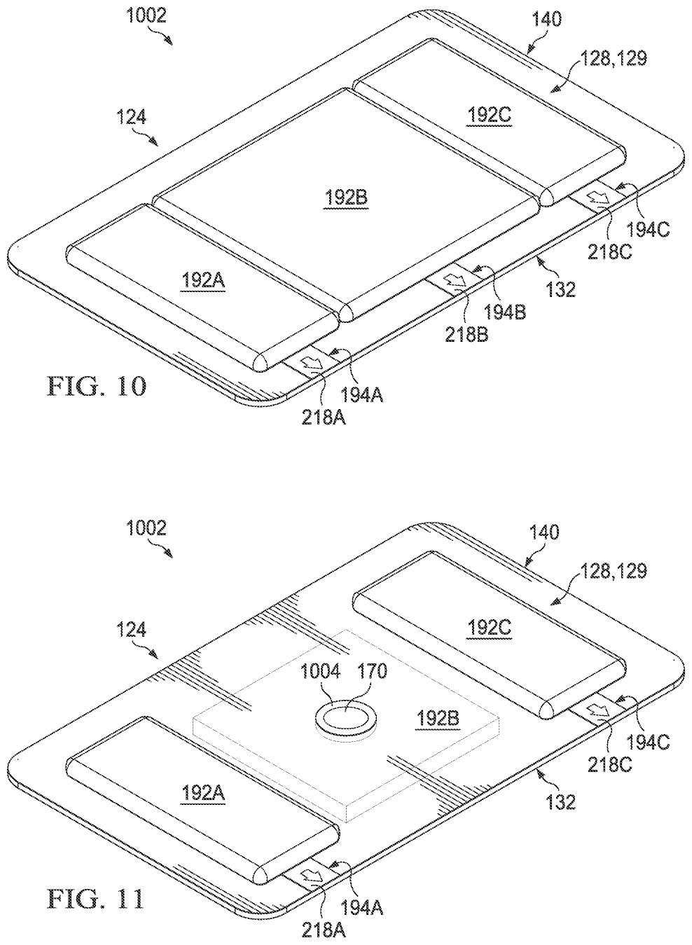

Referring to FIGS. 10 and 11, some illustrative example embodiments of a system 1002, analogous to the system 102, may include multiple pump modules 129 in fluid communication with the dressing 124. FIG. 10 shows the system 1002 with multiple pump modules 129 coupled to the dressing 124. For example, the pump 192 in the previously described system 102 may be a first pump 192A in the system 1002. The system 1002 may further include a second pump 192B and a third pump 192C. Further, the pump actuation assembly 194 in the previously described system 102 may be a first pump actuation assembly 194A in the system 1002 that may be associated with the first pump 192A. The system 1002 may further include a second pump actuation assembly 194B associated with the second pump 192B, and there may be a third pump actuation assembly 194C associated with the third pump 192C. The first pump actuation assembly 194A may have a pull tab 218A that can be used to activate the first pump 192A. The second pump actuation assembly 194B may have a pull tab 218B that can be used to activate the second pump 192B. The third pump actuation assembly 194C may have a pull tab 218C that can be used to activate the third pump 192C.

The second pump 192B may be larger than the first pump 192A and the third pump 192C such that the second pump 192B has a higher volume or capacity that the first pump 192A and the third pump 192C. The second pump actuation assembly 194B may be adhered to the dressing 124 with a releasable adhesive. The second pump 192B may be used to initially prime, evacuate, or draw down the dressing 124, for example, by removing excess air in effort to bring the dressing 124 down to a desired reduced pressure level. This may be when there is a large volume of air that needs to be removed from the tissue site 104 in order to reach the desired reduced pressure. After the tissue site 104 is at a desired pressure, the second pump 192B can be removed from the dressing 124 and discarded. FIG. 11 shows the system 1002 with the second pump 192B and the second pump actuation assembly 194B removed from the dressing 124. The first pump 192A and the third pump 192C may then be activated either at the same time or at different times to maintain the reduced pressure at the tissue site 104. When the second pump 192B and the second pump actuation assembly 194B are removed, the system 1002 may be less obtrusive for the user during the treatment duration.

The first pump 192A, the second pump 192B, and the third pump 192C may each be in fluid communication with the dressing 124 when activated. There may be apertures in the dressing 124, such as aperture 170, configured to enable fluid communication between the second pump 192B and the dressing 124. There may be similar apertures in the dressing 124 that allow fluid communication between the dressing 124 and the first pump 192A and the dressing 124 and the third pump 192C. In some embodiments, there may be a check valve 1004 disposed through or associated with the aperture 170 in the dressing 124. When the second pump 192B is activated and is drawing the tissue site 104 to the desired reduced pressure, the check valve 1004 may be open to enable fluid communication between the dressing 124 and the first pump 192A. When the second pump 192B is not activated, the check valve 1004 may be closed to prevent fluid from escaping from the dressing 124. The check valve 1004 may ensure that no fluid escapes through aperture 170 after the second pump 192B and the second pump actuation assembly 194B have been removed from the system 1002.

In operation of the system 102 according to some illustrative embodiments, the optional interface manifold 120 may be disposed against or proximate to the tissue site 104. The dressing 124 may then be applied over the interface manifold 120, if equipped, and the tissue site 104 to form the sealed space 174. Specifically, the base layer 132 may be applied covering the interface manifold 120 and/or the tissue surrounding the tissue site 104. The materials described above for the base layer 132 have a tackiness that may hold the dressing 124 initially in position. The tackiness may be such that if an adjustment is desired, the dressing 124 may be removed and reapplied. Once the dressing 124 is in the desired position, a force may be applied, such as by hand pressing, on a side of the scaling member 140 opposite the tissue site 104. The force applied to the sealing member 140 may cause at least some portion of the adhesive 136 to penetrate or extend through the plurality of apertures 160 and into contact with tissue surrounding the tissue site 104, such as the epidermis 106, to releaseably adhere the dressing 124 about the tissue site 104. In this manner, the configuration of the dressing 124 described above may provide an effective and reliable seal against challenging anatomical surfaces, such as an elbow or heal, at and around the tissue site 104. Further, the dressing 124 permits re-application or re-positioning to, for example, correct air leaks caused by creases and other discontinuities in the dressing 124 and the tissue site 104. The ability to rectify leaks may increase the reliability of the therapy and reduce power consumption.

As the dressing 124 comes into contact with fluid from the tissue site 104, the fluid moves through the apertures 160 toward the fluid management assembly 144. The fluid management assembly 144 wicks or otherwise moves the fluid through the optional interface manifold 120 and away from the tissue site 104. As described above, the interface manifold 120 may be adapted to communicate fluid from the tissue site 104 rather than store the fluid. Thus, the fluid management assembly 144 may be more absorbent than the interface manifold 120. The fluid management assembly 144 being more absorbent than the interface manifold 120 provides an absorbent gradient through the dressing 124 that attracts fluid from the tissue site 104 or the interface manifold 120 to the fluid management assembly 144. Thus, in some embodiments, the fluid management assembly 144 may be adapted to wick, pull, draw, or otherwise attract fluid from the tissue site 104 through the interface manifold 120. In the fluid management assembly 144, the fluid initially comes into contact with the first wicking layer 176. The first wicking layer 176 may distribute the fluid laterally along the surface of the first wicking layer 176 as described above for absorption and storage within the absorbent layer 184. Similarly, fluid coming into contact with the second wicking layer 180 may be distributed laterally along the surface of the second wicking layer 180 for absorption within the absorbent layer 184.

Once the dressing 124 is correctly placed at the tissue site 104, the pump module 129 may be activated. The pump module 129 can be activated by pulling at least one pull tab 218 to break the zone of weakness 214 of the film layer 210. Once the opening 215 is formed at the zone of weakness 214, the pump module 129 may create a reduced pressure at the dressing 124. When activated, the pump module 129 may draw fluids through the aperture 170 of the dressing 124 and through the passage 222 of the port 212. Fluid may then flow through the opening 208 of the pump housing 198 and into the biasing element 196. As fluid flows into the biasing element 196, the biasing element 196 may expand.

In some embodiments, the pump 192 may be re-primed once the biasing element 196 reaches its expanded state. The user can pinch the pump 192 to move the biasing element 196 from the expanded state to the compressed state. Once in the compressed state, the biasing element 196 can begin to expand and generate a reduced pressure at the dressing 124. This may allow the system 102 to remain at the tissue site 104 for an extended period of time.

Also provided are illustrative example methods for generating negative pressure. In some example embodiments, a method for generating negative pressure may include providing the dressing 124 which may be configured to be placed adjacent to the tissue site 104. The method may further include providing the reduced-pressure source 128, such as the pump module 129, which may be configured to be placed in fluid communication with the dressing 124 and to provide negative pressure to the dressing 124 when the pump module 129 is activated. The pump module 129 may include the pump 192 and the pump actuation assembly 194. The pump 192 of the pump module 129 may be activated by breaking the sacrificial seal 213 of the pump actuation assembly 194 to permit the pump 192 to draw fluids from the dressing 124 to the pump module 129. Breaking the sacrificial seal 213 of the pump actuation assembly 194 may include, for example, pulling at least one pull tab 218 of the actuation device 216 of the pump actuation assembly 194. Pulling at least one pull tab 218 may break the sacrificial seal 213 at a zone of weakness 214 to create the opening 215.

In some example embodiments, the method may further include coupling the port 212 of the pump actuation assembly 194 to the pump 192 and the dressing 124. The port 212 may be configured to enable fluid communication between the dressing 124 and the pump 192. In other embodiments, the method may further include coupling the port 212 of the pump actuation assembly 194 to the conduit 806. The conduit 806 may be configured to be coupled in fluid communication between the port 212 and the dressing 124. In some embodiments, the pump housing 198 of the pump 192 can be pinched after the pump 192 has reached an expanded state in order to re-activate the pump 192.

In some example embodiments, the pump 192 can be coupled to the pump actuation assembly 194 at the pump mounting portion 224 of the pump actuation assembly 194. The external mounting portion 226 of the pump actuation assembly 194 can be positioned opposite from the pump mounting portion 224. In some embodiments, the external surface of the external mounting portion 226 can carry a releasable adhesive. In some examples, the pump 192 can be the first pump 192A and the pump actuation assembly 194 can be the first pump actuation assembly 194A. The system 1002 may also include at least the second pump 192B and the second pump actuation assembly 194B. The first pump 192A or the second pump 192B can optionally be removed from the pump module after the dressing 124 is drawn to a desired negative pressure. If the second pump 192B is removed from the pump module 129, the first pump 192A can be activated before or after the second pump 192B is removed from the pump module 129, or at a suitable time when the dressing 124 is drawn to a desired negative pressure.

The systems, apparatuses, and methods described herein may provide significant advantages. For example, the system 102 does not have any rigid electrical pumps. More specifically, the pump 192 and the actuation device 216 are not electronic. The system 102 is soft, lightweight, unobtrusive, and quiet. The system 102 may provide less disruption to patient life which may result in higher treatment compliance. The system 102 may have embodiments that allow the reduced-pressure source 128 to be re-primed so that the system 102 can stay in place at a tissue site 104 for longer than the time it takes the biasing element 196 to go from the compressed state to the expanded state. Other embodiments may allow the reduced-pressure source 128 to be connected to the dressing 124 with a conduit 806 so the reduced-pressure source 128 can be located separately from the dressing 124. In other embodiments, there may be multiple pumps 192 to allow for more comfort to the user once at least one of the multiple pumps 192 is removed from the system 102. The system 102 may provide significant advantages since it is lighter, quieter, and less obtrusive than other currently available systems.

Although this specification discloses advantages in the context of certain illustrative, non-limiting embodiments, various changes, substitutions, permutations, and alterations may be made without departing from the scope of the appended claims. Further, any feature described in connection with any one embodiment may also be applicable to any other embodiment.

1. A system for negative pressure therapy, the system comprising:
a dressing configured to be placed adjacent to a tissue site;
a pump configured to be positioned in fluid communication with the dressing and to provide negative pressure to the dressing when activated, the pump comprising a biasing element and a pump housing; and
a pump actuation assembly comprising a sacrificial seal and an actuation device configured to activate the pump when the sacrificial seal is broken.

2. The system of claim 1, wherein the biasing element is configured to generate a variable volume within the pump housing when the pump is activated.

3. The system of claim 1, wherein the biasing element comprises foam and the pump housing comprises a film defining at least a portion of a sealed chamber that encases the foam.

4. The system of claim 3, wherein the foam is configured to move from a compressed state having a first volume prior to activation of the pump to an expanded state having a second volume after activation of the pump, and wherein the second volume is greater than the first volume.

5. The system of claim 1, wherein the sacrificial seal comprises a film layer including a zone of weakness and the actuation device comprises at least one pull tab configured to stretch the film layer to form an opening at the zone of weakness and to activate the pump when pulled.

6. The system of claim 5, wherein the at least one pull tab is coupled to the film layer or formed integrally with the film layer.

7. The system of claim 5, wherein the sacrificial seal is moveable from a sealed state to an activated state in which the pump is activated, and wherein the sacrificial seal is closed in the sealed state and open in the activated state, and wherein the opening is formed through the sacrificial seal in the activated state when the film layer is stretched to failure at the zone of weakness.

8. The system of claim 1, wherein the sacrificial seal is moveable from a sealed state to an activated state in which the pump is activated, wherein the sacrificial seal is closed in the sealed state and open in the activated state, wherein an opening is formed through the sacrificial seal in the activated state to activate the pump, and wherein the biasing element is configured to draw fluid into the pump housing through the opening when the pump is activated.

9. The system of claim 1, wherein the pump housing comprises at least one check valve.

10. (canceled)

11. The system of claim 1, further comprising a port defined through the pump actuation assembly and configured to enable fluid communication between the dressing and the pump.

12. The system of claim 11, wherein the sacrificial seal is positioned in the port of the pump actuation assembly.

13. The system of claim 11, wherein the sacrificial seal is positioned in the port and between a pump mounting portion and an external mounting portion of the pump actuation assembly, and wherein the pump mounting portion and the external mounting portion each comprise a ring of a closed cell foam.

14.-16. (canceled)

17. The system of claim 1, wherein the pump housing is configured to permit re-activation of the pump to maintain a desired negative pressure at the dressing.

18. The system of claim 1, wherein the pump housing comprises:
a sealed chamber configured to enclose the biasing element;
a base configured to be in fluid communication between the dressing and the sealed chamber; and
at least one exhaust port configured to provide fluid communication between the sealed chamber and ambient environment.

19. The system of claim 1, wherein the pump is coupled to the pump actuation assembly at a pump mounting portion of the pump actuation assembly, the pump actuation assembly further comprising an external mounting portion positioned opposite from the pump mounting portion, and wherein an exterior surface of the external mounting portion carries a releasable adhesive.

20. The system of claim 19, wherein the pump is a first pump and the pump actuation assembly is a first pump actuation assembly, the system further comprising at least a second pump and a second pump actuation assembly, wherein one of the first pump or the second pump are configured to be removed after the dressing is drawn down to a desired negative pressure.

21. A pump module, the pump module comprising:
a pump comprising a biasing element and a pump housing; and
a pump actuation assembly comprising a sacrificial seal and an actuation device configured to activate the pump when the sacrificial seal is broken.

22.-39. (canceled)

40. A method for generating negative pressure, the method comprising:
providing a dressing configured to be placed adjacent to a tissue site;
providing a pump module configured to be placed in fluid communication with the dressing and to provide negative pressure to the dressing when activated, the pump module comprising a pump and a pump actuation assembly; and
activating the pump by breaking a sacrificial seal of the pump actuation assembly to permit the pump to draw fluids from the dressing.

41. (canceled)

42. (canceled)

43. The method of claim 40, wherein breaking the sacrificial seal of the pump actuation assembly comprises pulling at least one pull tab of an actuation device of the pump actuation assembly to break the sacrificial seal at a zone of weakness.

44. The method of claim 40, further comprising pinching a pump housing of the pump after the pump has reached an expanded state to re-activate the pump.

45. (canceled)

46. (canceled)

47. The method of claim 40, wherein the pump is a first pump and the pump actuation assembly is a first pump actuation assembly, the method further comprising providing at least a second pump and a second pump actuation assembly, further comprising removing the first pump from the pump module after the dressing is drawn to a desired negative pressure.

48. (canceled)

49. The method of claim 47, further comprising activating the second pump after the first pump is removed from the pump module.
